(19) United States
(12) Patent Application Publication
(10) Pub. No.: US 2025/0318717 A1
(43) Pub. Date: Oct. 16, 2025

(54) SHAFT PURGING SYSTEM FOR ENDOSCOPE

(71) Applicant: GYRUS ACMI, INC. D/B/A OLYMPUS SURGICAL TECHNOLOGIES AMERICA, Westborough, MA (US)

(72) Inventors: Kester Julian Batchelor, Mound, MN (US); David M. Leak, Brooklyn Park, MN (US)

(21) Appl. No.: 19/169,463

(22) Filed: Apr. 3, 2025

Related U.S. Application Data
(60) Provisional application No. 63/632,451, filed on Apr. 10, 2024.

(51) Int. Cl. A61B 1/015; A61B 1/12; A61B 17/22; A61B 90/00; A61M 1/00

(52) U.S. Cl. CPC A61B 1/015; A61B 1/126; A61B 17/2202; A61M 1/82; A61B 2017/22079; A61B 2090/064; A61B 2217/007

(57) ABSTRACT

A purging device for a medical instrument. The medical instrument can include a handle, a shaft extending from the handle, and suction tubing extending between the handle and a suction source. The purging device can include a housing having a chamber defining a volume of fluid and an attachment interface configured to attach the purging device to the handle of the medical instrument such that the chamber fluidically connects the suction source and the shaft. The purging device can also include an engagement member that can be engageable from an exterior of the housing. The engagement member can be configured to generate a positive pressure pulse within the chamber to energize the volume of fluid from the chamber and unclog debris from either one of the suction tubing or the shaft of the medical instrument.

### CLAIM OF PRIORITY

This patent application claims the benefit of priority, under 35 U.S.C. Section 119(e), to Kester Batchelor U.S. Patent Application Ser. No. 63/632,451, entitled “SHAFT PURGING SYSTEM FOR ENDOSCOPE,” filed on Apr. 10, 2024, (Attorney Docket No. 5409.894PRV), which is hereby incorporated by reference herein in its entirety

### TECHNICAL FIELD

Examples described herein generally relate to an endoscope and, more specifically, to an endoscope, including a shaft purging system.

### BACKGROUND

The field of medical endoscopy has seen significant advancements over the years, leading to the development of various devices and systems that assist clinicians in diagnostic and therapeutic procedures. Endoscopes are medical instruments that are inserted into the body to observe or perform medical procedures in the interior of a hollow organ or cavity. During medical procedures, maintaining clear visibility and functionality of the endoscope aids the medical professional in the navigation and operation of the endoscope.

One of the challenges encountered during endoscopic procedures is the accumulation of debris within the working channels of the endoscope, such as the suction channel. This debris can include tissue fragments, blood, and other matter that can obstruct the channels, leading to reduced efficiency in debris removal and potential complications during the procedure. The small diameters of the working channels in endoscopes make them particularly susceptible to such blockages.

### SUMMARY

In examples, an endoscope can include a console including control circuitry for controlling operation of the endoscope during a medical procedure; a suction source to provide suction to the endoscope; a handle configured to be held by a clinician to control the endoscope during the medical procedure; a shaft extending from the handle; a suction line routed at least partially within the handle to fluidically connect the shaft to the suction source; and a purging device fluidically connected to the suction line between the suction source and the shaft, the purging device configured to generate a positive pressure pulse to clear debris accumulated within either the shaft or the suction line.

In examples, a purging device for a medical instrument, the medical instrument including a handle, a shaft extending from the handle, and suction tubing extending between the handle and a suction source, the purging device including: a housing including: a chamber defining a volume of fluid; and an attachment interface configured to attach the purging device to the handle of the medical instrument such that the chamber fluidically connects the suction source and the shaft; and an engagement member engageable from an exterior of the housing, the engagement member configured to generate a positive pressure pulse within the chamber to energize the volume of fluid from the chamber and unclog debris from either one of the suction tubing or the shaft of the medical instrument.

### BRIEF DESCRIPTION OF THE DRAWINGS

Various examples are illustrated in the figures of the accompanying drawings. Such examples are demonstrative and not intended to be exhaustive or exclusive examples of the present subject matter.

FIG. 1 illustrates an example diagram of an example endoscope including an example system for fluid management.

FIG. 2 illustrates an example medical instrument including an example system for fluid management

FIG. 3 illustrates a cross-sectional view of an example of a purging device.

FIG. 4 illustrates a cross-sectional view of an example of the purging device of FIG. 3 in an engaged state.

FIG. 5 illustrates a cross-sectional view of an example of a purging device.

FIG. 6 illustrates a cross-sectional view of an example of the purging device of FIG. 5 in an engaged state.

FIG. 7 illustrates a perspective view of an example of a purging device.

FIG. 8 illustrates a cross-sectional view of an example of a purging device.

FIG. 9 illustrates a cross-sectional view of an example of the purging device from FIG. 8 in an engaged state.

FIG. 10 is a schematic diagram of an exemplary computer-based clinical decision support system (CDSS).

FIG. 11 is a block diagram illustrating an example of a machine upon which one or more examples may be implemented.

FIG. 12 illustrates a cross-sectional view of an example of a purging device.

FIG. 13 illustrates a cross-sectional view of an example of a purging device.

### DETAILED DESCRIPTION

Medical instruments (e.g., a sheath, an endoscope, or the like) can include a tubular portion or a shaft (elongated member) insertable into an interior of an organ or a cavity (or lumen) of the body to assist in diagnosis or treatment (e.g., removal of stones or tissue from within the patient). One or more working channels (e.g., a suction channel or an irrigation channel) can be disposed inside and extend along a length of the tubular portion. To lower the risk of unintentionally engaging with unintended tissue, the insertable tubular portion can have a smaller diameter than the rest of the endoscope. Consequently, the working channels can also have small lumen diameters. Therefore, because of the small diameters of the working channels, tissue debris and foreign objects (e.g., calculi and fragments thereof) can accumulate and clog the working channel.

A fluid management system can be connected to the medical instrument to provide an irrigation fluid (e.g., saline) and suction to the endoscope. The fluid management system can include inflow tubing and outflow tubing fluidically connected to the irrigation channel and the suction channel of the endoscope, respectively. Similar to the working channels, the inflow tubing and the outflow tubing can become clogged, kinked, or blocked.

In this document, “clog” refers to tissue debris, calculi (e.g., kidney stones or stone fragments), and other matter that can accumulate and block the lumen of a channel partially or completely, “clogging” refers to a state of partial or complete blockage of the channel lumen, and “kink” refers to tubing bending, warping, or becoming deformed such as to interfere with fluid or debris flow therethrough.

Clogging in a shaft, the suction channel, or the outflow tubing can significantly reduce the efficiency of removing tissue debris and stone fragments therethrough. Delayed or inefficient removal of unwanted matters from the anatomical site can inhibit or prevent further treatment (e.g., debridement or ablation of stones), contaminate the anatomical site, or expose the patient to an increased risk.

Various approaches have been attempted to prevent or resolve channel clogging in medical instruments. For example, breaking unwanted matters (e.g., tissue debris or stone fragments) into finer pieces can reduce the likelihood of being clogged in the channel. This, however, can consume more energy, take a longer procedure time, and potentially increase patient risk due to the added procedure complexity and time. Fine particles or stone dust can reduce the visibility of the surgical field. Conventionally, unclogging is usually performed externally, which can require a clinician to retract the scope from the body, flush the obstructed scope to unclog it, and insert it back into the anatomical site. This approach increases procedure time, adds inconvenience to the clinician, and can increase surgical risks for the patient.

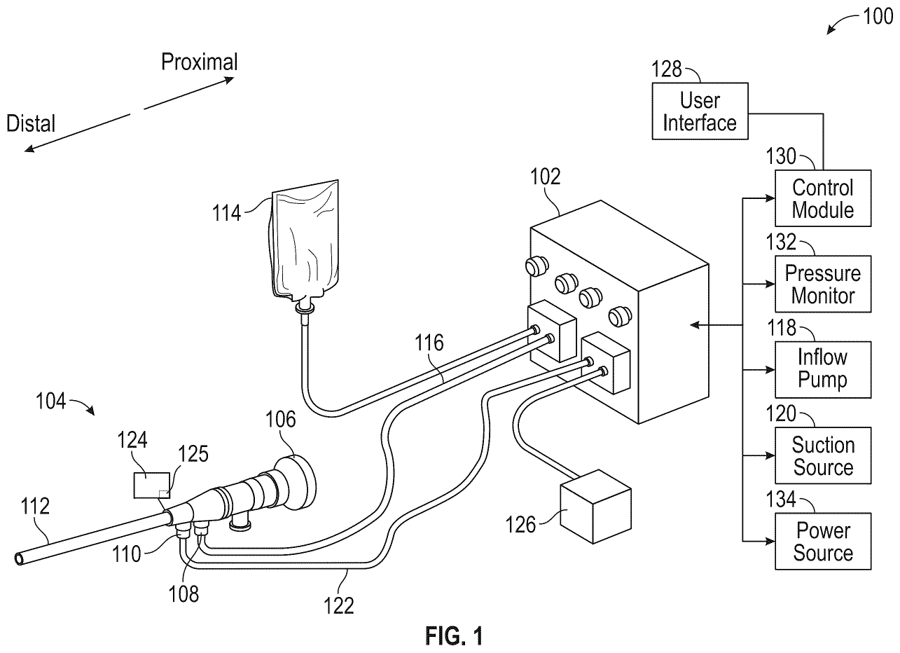

The above discussion is intended to provide an overview of the subject matter of the present disclosure. It is not intended to provide an exclusive or exhaustive explanation of the disclosure. The description below is included to provide further information about the present patent application. The present disclosure relates to medical instruments used to remove stones or tissue from within a patient. Thus, many medical instruments can use the systems, methods, and devices discussed herein. For clarity and brevity, FIGS. 1-13 will focus on an implementation of the present disclosure as it relates to an endoscope. However, as discussed herein, the present disclosure can be implemented to other medical instruments used to remove debris or tissue from within a patient.

FIG. 1 illustrates an example diagram of an example endoscopic system 100 including an example system for fluid management. The endoscopic system 100 can be a lithotripter configured to remove debris from within a kidney of a patient, an ablation device to remove debris from within a patient, any other medical instrument configured to be inserted within the patient, or the like. The endoscopic system 100 can include a console 102, an endoscope 104, a handle 106, an irrigation fluid port 108, a suction port 110, a shaft 112, an irrigation fluid source 114, an irrigation line 116, an inflow pump 118, a suction source 120, a suction line 122, a purging device 124, a debris collector 126, a user interface 128, a control module 130, a pressure monitor 132, and a power source 134.

The console 102 can include control circuitry (e.g., the control module 130, the pressure monitor 132, or the like) for controlling the operation of the endoscopic system 100 during a medical procedure. The console 102 can be fluidically connected to the irrigation fluid source 114, the inflow pump 118, the suction source 120, and the debris collector 126. The console 102 can also be in communication with the user interface 128, which can be a standalone device, or integral to the console 102.

The endoscope 104 can be in communication with the console 102 such that the console 102 can control the operation of the endoscope 104. The endoscope 104 can include the handle 106 and the shaft 112. The handle 106 can be configured to be held by the clinician to control the endoscope 104 during the medical procedure. The handle 106 can be generally elongated such as to improve the ergonomics of the clinician during the use of the endoscope 104.

The handle 106 can include the irrigation fluid port 108 and the suction port 110. The irrigation fluid port 108 can fluidically connect the irrigation fluid source 114 to the shaft 112 to provide irrigation fluid (e.g., water, saline, other biocompatible liquids, or the like) from the irrigation fluid source 114 and to the procedure site (e.g., the distal end of the shaft 112). The inflow pump 118 can be configured to provide the irrigation fluid source 114 to the distal end of the shaft 112 via the irrigation line 116 and the irrigation fluid port 108. The suction port 110 can fluidically connect the shaft 112 to the debris collector 126 via the suction line 122 to draw debris, fluid, excess irrigation fluid, or the like away from the distal end of the shaft 112. The suction source 120 can provide suction to the endoscopic system 100 such as to draw debris (e.g., kidney stone fragments, ablated tissue, blood, excess irrigation fluid, or the like) away from the distal end of the shaft 112 and toward the debris collector 126 via the suction port 110 and the suction line 122. The suction source 120 can be a pump configured to draw the debris away from the distal end of the shaft 112. The debris collector 126 can be configured to measure collected debris during the medical procedure.

The shaft 112 can extend from the handle 106 and can be fluidically connected to the handle 106. The shaft 112 can include one or more lumens (e.g., a source fluid lumen and a suction lumen), which can be fluidically connected to the irrigation fluid port 108 and the suction port 110, respectively, to provide irrigation fluid from the irrigation fluid source 114 to the distal end of the shaft 112 and draw debris, fluid, or the like away from the distal end of the shaft 112. Thus, the irrigation line 116 can be routed at least partially within the handle 106 to fluidically connect a distal end of the shaft 112 to the irrigation fluid source 114 and the suction line 122 can be routed at least partially within the handle 106 to fluidically connect the shaft 112 to the suction source 120.

The purging device 124 can be fluidically connected to the suction line 122 between the suction source 120 and the shaft 112. The purging device 124 can be configured to generate a positive pressure pulse to clear debris accumulated within either the shaft 112 or the suction line 122. In examples, the purging device 124 can be completely enclosed by the handle 106, can at least partially extend outside the handle 106, or can be completely outside of the handle 106.

The control module 130 can be configured to operate the endoscopic system 100, and more specifically, the endoscope 104 and the purging device 124, based on information from at least one of the user via the user interface 128, a pressure within the system detected by the pressure monitor 132, pump readings from either of the inflow pump 118 or the suction source 120, power readings from the power source 134, or any other component of the endoscopic system 100. In examples, the control module 130 can generate control signals to alter operational parameters of the inflow pump 118 to alter the amount of irrigation fluid from the irrigation fluid source 114 flowing to the distal end of the shaft 112. The control module 130 can send a control signal to increase a suction provided to the endoscopic system 100 via the suction source 120 to increase an amount of fluid or debris being drawn from the distal end of the shaft 112. The control module 130 can send a control signal to operate the purging device 124 to help clear any clogs, kinks, blockages, or any combination thereof in the shaft 112 or the suction line 122.

In examples, the control module 130 can receive a signal from a sensor 125 within the purging device 124. The signal from the sensor 125 of the purging device 124 can be indicative of the actuation of the purging device 124. The control module 130 can transmit, based on the detected actuation of the purging device 124, an activation summary. The activation summary can be configured to notify the clinician that the purging device 124 is activated and include one or more parameters detected by sensors of the endoscopic system 100, such as the pressure from the pressure monitor 132, a pump rate, e.g., a voltage, from inflow pump 118, a pump rate, e.g., a voltage the suction source 120, or the like. For example, the activation summary can be transmitted to the user interface 128 to provide the clinician with a pressure within the endoscope during the activation of the purging device 124 and a current pressure within the endoscope. The current pressure can be live to show the effectiveness of the activation of the purging device 124.

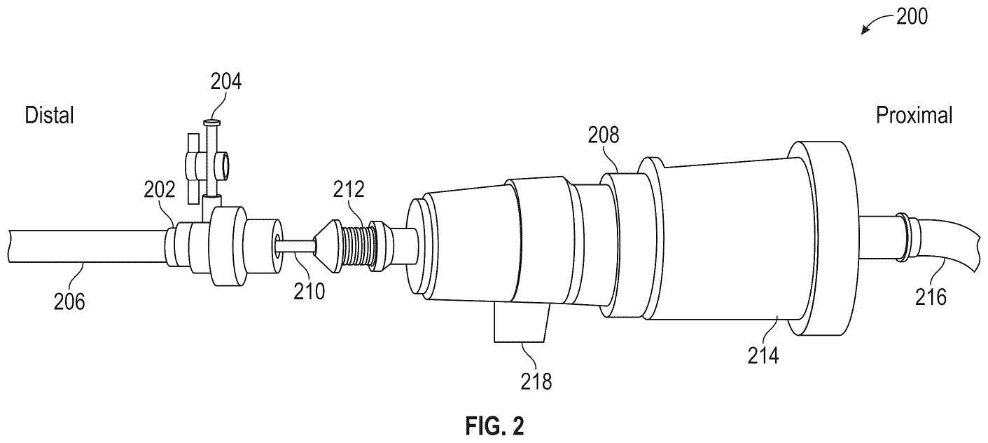

FIG. 2 illustrates an example medical instrument 200 including an endoscope 202 (e.g., endoscope 104 (FIG. 1)), a stone ablation device 208, and a purging device 218 (e.g., purging device 124 (FIG. 1)). The endoscope 202 can include an irrigation fluid port 204 (e.g., irrigation fluid port 108 (FIG. 1)) and an elongated member 206 (e.g., shaft 112 (FIG. 1)).

The medical instrument 200 can be configured to remove debris (e.g., for example, stones) from a target area within a patient. As such, the medical instrument 200 can receive irrigation fluid via the irrigation fluid port 204, which can be directed to a distal end of the elongated member 206 and toward the target area to help with the breaking and extraction of stones from the target area.

The stone ablation device 208 can include a shaft 210, vibration source 212, collection chamber 214, and a suction port 216 (e.g., suction port 110 (FIG. 1)). The shaft 210 can be configured to be inserted within the elongated member 206 such as to direct vibrations generated by the vibration source 212 toward a distal end of the elongated member 206. A suction source (e.g., suction source 120 FIG. 1) can be connected to the medical instrument 200 via the suction port 216 to provide suction from the distal end of the elongated member 206, through the irrigation fluid port 204 and the stone ablation device 208. Stones can be drawn through the shaft 210 and toward the collection chamber 214 and the suction port 216. In examples, the stones can be collected within the collection chamber 214, and the leftover fluids and smaller debris can continue through the collection chamber 214 and into the suction port 216.

Stones, fragments of stones, other debris, or combinations thereof can become lodged within the endoscope 202 or the stone ablation device 208 such as to prevent irrigation fluid or debris from being drawn through the endoscope 202 or the stone ablation device 208. Thus, the medical instrument 200 includes the purging device 218, which can be integral to the stone ablation device 208, attached to the stone ablation device 208, or connected to both of the endoscope 202 and the stone ablation device 208. The purging device 218 can be configured to generate a positive pressure pulse to clear debris accumulated within any of the elongated member 206, the shaft 210, or within the endoscope 202 or the stone ablation device 208.

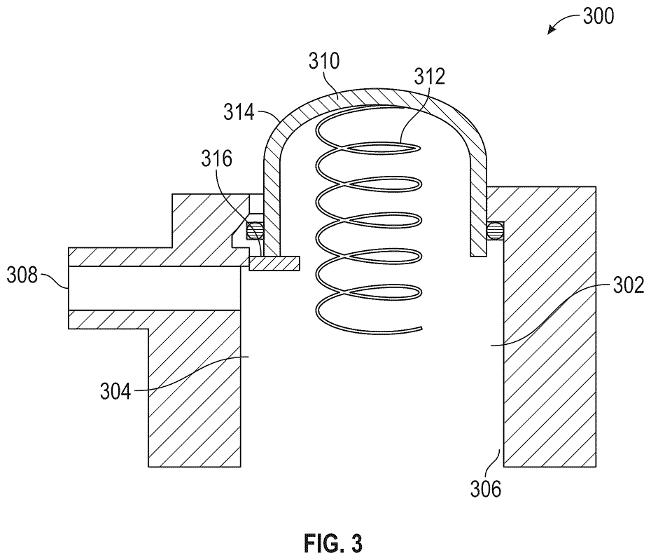

FIG. 3 illustrates a cross-sectional view of an example of a purging device 300 (e.g., the purging device 124 (FIG. 1)). The purging device 300 can be installed within the handle 106 (FIG. 1). In examples, the purging device 300 can be installed within the handle 106 such that a portion of the purging device 300 (e.g., an elastic member 310) extends outside of the handle 106 such that an end-user can engage the elastic member 310. The purging device 300 can include a suction chamber (e.g., a chamber 302) and a sealing member (e.g., an elongated member 316). In FIG. 3, the purging device 300 is shown in starting position 314.

The chamber 302 can be configured to hold a volume of the fluid 304. The fluid 304 can be drawn from the distal end of the shaft 112 (FIG. 1) by the suction source 120, and can include irrigation fluid (e.g., from the irrigation fluid source 114), blood, surgical debris (e.g., ablated tissues, whole or fragments of nephrolithiasis (e.g., renal calculi, urolithiasis, or the like)), or any combination thereof. The chamber 302 can be fluidically connected to the suction line (e.g., the suction line 122 (FIG. 1) between the suction source (e.g., the suction source 120 (FIG. 1) or the debris collector 126 (FIG. 1)) and the shaft (e.g., the shaft 112 (FIG. 1)). The debris within the fluid 304 can become clogged, stuck, or can cause blockages of the fluid 304 from the distal tip of the shaft 112 toward the debris collector 126 or the suction source 120. Such blockages can prevent a medical professional from being able to draw fluid or debris from the distal end of the shaft 112, which can result in poor imaging, poor surgical results, or the like. As discussed above, the chamber 302 can be disposed of within the handle 106 (FIG. 1) or can be external to the handle 106. The chamber 302 can include a shaft port (e.g., an inlet 306), a suction port (e.g., an outlet 308), an elastic member 310, and a biasing member 312.

The inlet 306 can be fluidically connected to the shaft 112 (FIG. 1). As such, the fluid 304 (e.g., and any combination of debris) can flow from the distal end of the shaft 112 and into the chamber 302 via the inlet 306. The outlet 308 can be fluidically connected to the suction source (e.g., the suction source 120 (FIG. 1)) such that fluid from the distal end of the shaft 112 can flow to the suction source 120 via the inlet 306, the chamber 302, the outlet 308 and the suction line 122 (FIG. 1).

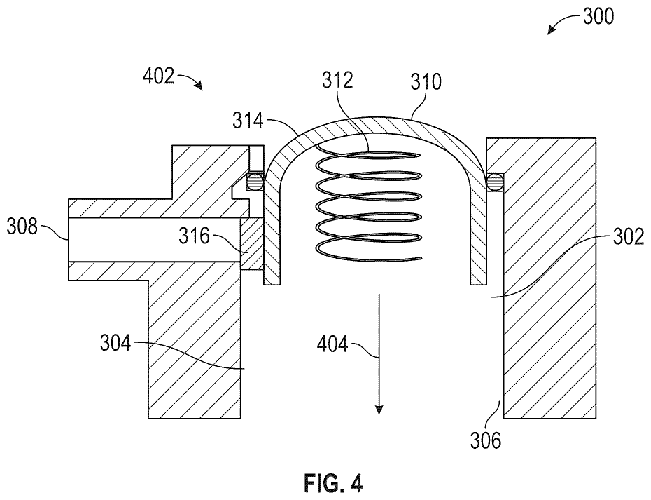

The elastic member 310 can be engageable (e.g., by an end-user of the endoscopic system 100 (FIG. 1)) to generate a positive pressure pulse (e.g., positive pressure pulse 404 (FIG. 4) within the chamber 302. The elastic member 310 can be disposed within the purging device 300 such that at least a portion of the elastic member 310 can be engaged by the end-user of the endoscopic system 100 (FIG. 1). For example, at least a portion of the elastic member 310 can extend outside of the handle 106 (FIG. 1)). The biasing member 312 (e.g., a spring) can be disposed within the chamber 302. The biasing member 312 can be configured to return the elastic member 310 to the starting position 314 after actuation of the biasing member 312 is complete.

The elongated member 316 can extend from the biasing member 312 within the chamber 302 such that the elongated member 316 translates within the chamber 302 as the elastic member 310 is engaged (e.g., by an end user of the endoscopic system 100) such that the elongated member 316 blocks the outlet 308 to direct the positive pressure pulse through the inlet 306 and toward the shaft (e.g., the shaft 112 (FIG. 1)).

FIG. 4 illustrates a cross-sectional view of an example of the purging device 300 of FIG. 3 in an engaged state 402. As discussed herein, moving the purging device 300 from the starting position 314 to the engaged state 402 can move the fluid 304 within the chamber 302 to generate a positive pressure pulse 404.

As discussed with reference to FIG. 3, the elongated member 316 can be connected to (e.g., or engageable by) the elastic member 310 such that the elongated member 316 translates as the elastic member 310 is engaged by the end-user of the endoscopic system 100 (FIG. 1). As the elongated member 316 translates, the elongated member 316 can cover the outlet 308 to prevent the fluid 304 within the chamber 302 from going out the outlet 308 and toward the debris collector 126. Because the outlet 308 is blocked by the elongated member 316 when the elastic member 310 is engaged by the end user, the positive pressure pulse 404 is directed toward the inlet 306 to provide energy into the suction line 122 (FIG. 1) and toward the distal end of the shaft 112 (FIG. 1). As such, the positive pressure pulse 404 can dislodge any debris that is clogged, or otherwise blocking, the shaft 112 (FIG. 1) or the suction line 122 (FIG. 1) of the endoscopic system 100 (FIG. 1).

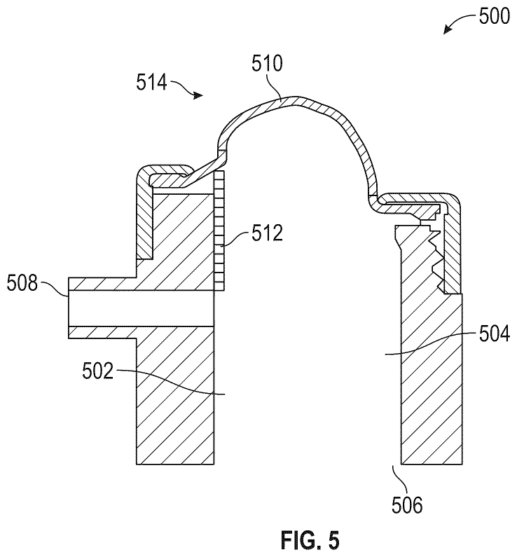

FIG. 5 illustrates a cross-sectional view of an example of a purging device 500. The purging device 500 can be installed within the handle 106 (FIG. 1). In examples, the purging device 500 can be installed within the handle 106 such that a portion of the purging device 500 (e.g., a flexible diaphragm 510) extends outside of the handle 106 such that an end-user can engage the flexible diaphragm 510. The purging device 500 can include a fluid chamber 502 configured to hold a fluidic volume (e.g., a fluid 504), a suction inlet 506, a suction outlet 508, a flexible diaphragm 510, and a blocking member 512. As shown in FIG. 5, the purging device 500 can be positioned in a resting position 514.

The fluid chamber 502 can be configured to hold a fluidic volume of the fluid 504. The fluid 504 can be drawn from the distal end of the shaft 112 (FIG. 1) by the suction source 120 (FIG. 1) and can include irrigation fluid (e.g., from the irrigation fluid source 114), blood, surgical debris (e.g., ablated tissues, whole or fragments of nephrolithiasis (e.g., renal calculi, urolithiasis, or the like)), or any combination thereof. The fluid chamber 502 can be fluidically connected to the suction line (e.g., the suction line 122 (FIG. 1) between the suction source (e.g., the suction source 120 (FIG. 1) or the debris collector 126 (FIG. 1)) and the shaft (e.g., the shaft 112 (FIG. 1)). The debris within the fluid 504 can become clogged, stuck, or can cause blockages of the fluid 504 from the distal tip of the shaft 112 toward the debris collector 126 or the suction source 120. Such blockages can prevent a medical professional from being able to draw fluid or debris from the distal end of the shaft 112, which can result in poor imaging, poor surgical results, or the like. As discussed above, the fluid chamber 502 can be disposed of within the handle 106 (FIG. 1) or can be external to the handle 106. The fluid 504 can include the suction inlet 506, the suction outlet 508, flexible diaphragm 510, and the blocking member 512.

The suction inlet 506 can fluidically connect the fluid chamber 502 to the shaft 112 (FIG. 1). The fluid 504 can flow from the distal tip of the shaft 112 and into the fluid chamber 502 via the suction inlet 506. The suction outlet 508 can be fluidically connected between the fluid chamber 502 and the suction source 120 (FIG. 1) or the debris collector 126 (FIG. 1). Thus, the distal end of the shaft 112 can be fluidically connected to the debris collector 126 such that the fluid 504 can flow from the distal tip of the shaft 112 and into the suction inlet 506, through the fluid 504, out the suction outlet 508 and toward the suction source 120 or the debris collector 126 via the suction line 122.

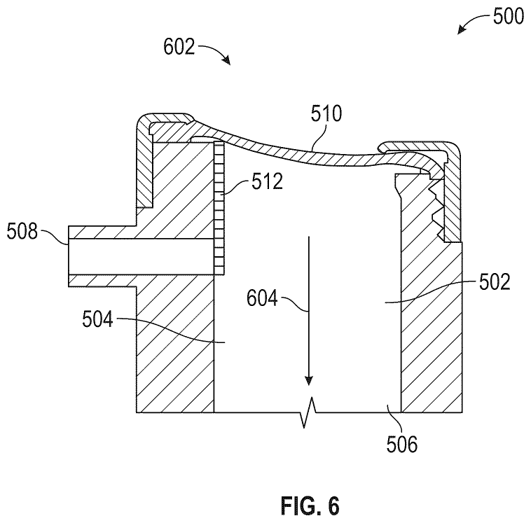

The flexible diaphragm 510 can define at least a portion of the fluid chamber 502 and can be engageable (e.g., by an end-user of the endoscopic system 100 (FIG. 1)) to generate a positive pressure pulse (e.g., positive pressure pulse 604 (FIG. 6)) within the fluid chamber 502. The flexible diaphragm 510 can be disposed of within the purging device 500 such that at least a portion of the flexible diaphragm 510 can be engaged by the end-user of the endoscopic system 100. For example, at least a portion of the flexible diaphragm 510 can extend outside of the handle 106 (FIG. 1)). The flexible diaphragm 510 can include shape memory material (e.g., material with elastic properties) such that the flexible diaphragm 510 returns to the resting position 514 when the flexible diaphragm 510 is not engaged by an end user of the endoscopic system 100. In examples, the volume of fluid 504 within the fluid chamber 502 can provide pressure within the fluid chamber 502 to help encourage the flexible diaphragm 510 toward the resting position 514.

FIG. 6 illustrates a cross-sectional view of an example of the purging device 500 of FIG. 5 in an engaged position 602. As discussed herein, moving the purging device 500 from the resting position 514 to the engaged position 602 can move the fluid 504 within the fluid chamber 502 to generate a positive pressure pulse 604.

As discussed herein with reference to FIG. 5, the blocking member 512 can be engageable by the flexible diaphragm 510 such that the blocking member 512 translates as the flexible diaphragm 510 is engaged by the end-user of the endoscopic system 100 (FIG. 1). As the blocking member 512 translates, the blocking member 512 can cover the suction outlet 508 to prevent the fluid 504 within the fluid chamber 502 from going out the suction outlet 508 and toward the suction source 120 or the debris collector 126. Because the suction outlet 508 is blocked by the blocking member 512 when the flexible diaphragm 510 is engaged by the end user, the positive pressure pulse 604 is directed toward the suction inlet 506 to provide energy into the suction line 122 (FIG. 1), and toward the distal end of the shaft 112 (FIG. 1). As such, the positive pressure pulse 604 can dislodge any debris that is clogged, or otherwise blocking, the shaft 112 (FIG. 1) or the suction line 122 (FIG. 1) of the endoscopic system 100 (FIG. 1).

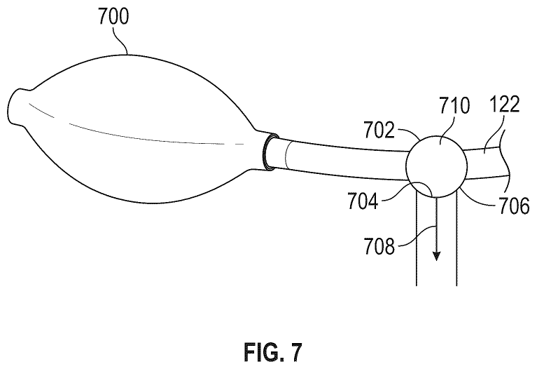

FIG. 7 illustrates a perspective view of an example of a purging device (e.g., a hand-actuated pressure bulb 700). The hand-actuated pressure bulb 600 can be external to the handle 106 (FIG. 1) or can extend such that at least a portion of the hand-actuated pressure bulb 600 extends within the handle 106. The hand-actuated pressure bulb 600 can be fluidically connected to the suction line 122 via a valve 702. The valve 702 can be configured to block fluid flow into the suction line (e.g., toward either of the suction source 120 (FIG. 1) or the debris collector 126 (FIG. 1)) when the hand-actuated pressure bulb 600 is actuated by an end-user. As shown in FIG. 6, the valve 702 can include a suction inlet 704 and a suction outlet 606. The suction inlet 704 can fluidically connect the valve 702 to the distal end of the shaft 112 (FIG. 1) via the suction line 122. The suction outlet 606 can fluidically connect the valve 702 to the suction source 120 (FIG. 1) or the debris collector 126 (FIG. 1) via the suction line 122.

During standard operation, a fluid (e.g., fluid 710) can flow freely through the valve 702 from the suction inlet 704, out the suction outlet 706, and toward the suction source 120 or the debris collector 126 via the suction line 122. If a blockage is detected in any of the suction line 122, the shaft 112, or the suction inlet 704, the hand-actuated pressure bulb 700 can be engaged by an end-user of the endoscopic system 100 (FIG. 1) to generate a positive pressure pulse 708. The valve 702 can be configured to block the suction outlet 706 in response to the positive pressure pulse 708 such as to direct the positive pressure pulse 708 through the suction inlet 704 and toward the distal end of the shaft 112. As such, the positive pressure pulse 708 can dislodge any debris that is clogged, or otherwise blocking, the suction inlet 704, the shaft 112 (FIG. 1) or the suction line 122 (FIG. 1) of the endoscopic system 100 (FIG. 1).

In examples, the valve 702 can be configured to block the suction outlet 706 using a mechanical restraint (e.g., a block valve), a butterfly valve (e.g., activated as the hand-actuated pressure bulb 700 is engaged by the end-user), an actuated valve (e.g., activated as the hand-actuated pressure bulb 700 is engaged by an end user), a globe valve, or the like. In examples, the control module 130 can be configured to activate, or generate an alert such as to direct an end-user to activate the hand-actuated pressure bulb 700 upon detecting high pressure between the valve 702 and the distal end of the shaft 112 (FIG. 1). The control module 130 can also send a controlling signal to the valve 702 to alter fluid flow within the valve 702 and block the flow of fluid toward the suction source 120 or the debris collector 126 to direct the fluid through the suction outlet 706 and toward the distal tip of the shaft 112 (FIG. 1).

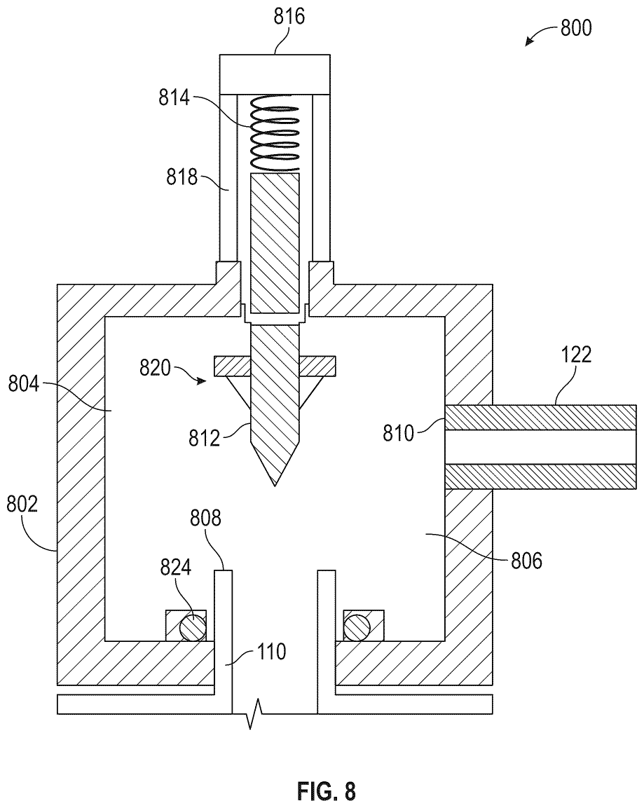

FIG. 8 illustrates a cross-sectional view of an example of a purging device 800. The purging device 800 can be installed within the handle 106 (FIG. 1). In examples, the purging device 800 can be installed within the handle 106 such that a portion of the purging device 800 (e.g., the engagement surface 816) extends outside of the handle 106 such that an end-user can engage the engagement surface 816. The purging device 800 can include a housing 802 including an attachment interface 824 and defining a suction chamber 804 that can be filled with a fluid 806 (e.g., fluid 304, fluid 504, or fluid 710), a shaft port 808, a suction port 810, a plunger 812, a biasing member 814, an engagement surface 816, and an engagement housing 818. The purging device 800, as shown in FIG. 8 is in the resting position 820).

The housing 802 can define the suction chamber 804 and can be configured to attach the purging device 800 to the endoscope 104 via the attachment interface 824. As shown in FIG. 8, the purging device 800 can be attached to the handle 106 between the suction port 110 and the suction line 122. The attachment interface 824 can include seals to prevent leaks during the operation of the purging device 800. Each purging device (e.g., the purging device 124, the purging device 300, the purging device 500, and the hand-actuated pressure bulb 700) can be similarly attached to the endoscope 104 such that the purging device fluidically connects the suction port 110 to the suction line 122 toward the suction source 120 or the debris collector 126.

The suction chamber 804 can be configured to hold a volume of the fluid 806. The fluid 806 can be drawn from the distal end of the shaft 112 (FIG. 1) by the suction source 120, and can include irrigation fluid (e.g., from the irrigation fluid source 114), blood, surgical debris (e.g., ablated tissues, whole or fragments of nephrolithiasis (e.g., renal calculi, urolithiasis, or the like)), or any combination thereof. The suction chamber 804 can be fluidically connected to the suction line (e.g., the suction line 122 (FIG. 1)) and the shaft (e.g., the shaft 112 (FIG. 1)). The debris within the fluid 806 can become clogged, stuck, or can cause blockages of the fluid 806 from the distal tip of the shaft 112 toward the debris collector 126 or the suction source 120. Such blockages can prevent a medical professional from being able to draw fluid or debris from the distal end of the shaft 112, which can result in poor imaging, poor surgical results, or the like.

The shaft port 808 can be fluidically connected to the shaft 112 (FIG. 1) such that the shaft port 808 fluidically connects the suction chamber 804 to the distal end of the shaft 112 via the suction line 122 (FIG. 1). The suction port 810 can be fluidically connected to the suction source (e.g., the suction source 120 (FIG. 1)) such that fluid from the distal end of the shaft 112 be drawn into the suction line 122, into the suction chamber 804 via the shaft port 808 and back into the suction line 122 toward the suction source 120 or the debris collector 126 via the suction port 810.

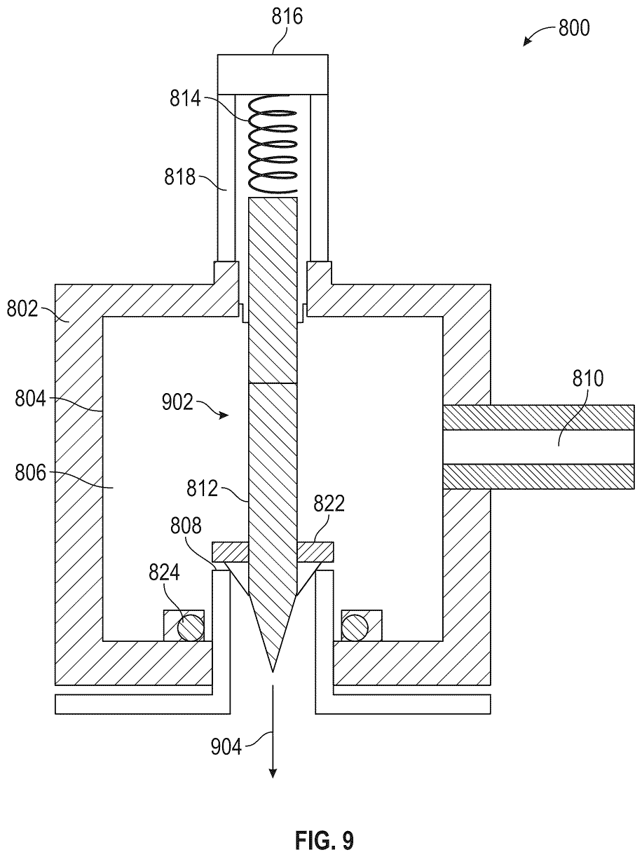

The plunger 812 can extend into the suction chamber 804. The plunger 812 can be configured to displace a volume of the fluid 806 to generate a positive pressure pulse (e.g., positive pressure pulse 904 (FIG. 9)) when actuated (e.g., moved from the resting position 820 to the working position 902).

The purging device 800 can include the engagement housing 818 and the engagement surface 816. The engagement housing 818 d the engagement surface 816 can contain the plunger 812, and a biasing member 814. The engagement surface 816 can be configured to be engaged by a user of the endoscopic system 100 (FIG. 1) to operate the purging device 800 and operate the purging device 800 between the resting position 820 to the working position 902 to generate the positive pressure pulse 904.

The biasing member 814 can be disposed of within the engagement surface 816 and the engagement housing 818 such that the biasing member 814 connects the engagement surface 816 to the plunger 812. The biasing member 814 can be configured to return the plunger 812 to the starting resting position 820 after actuation (e.g., after the end user stops engaging the engagement surface 816). In other words, the biasing member 814 can be configured to pull the plunger 812 toward the engagement surface 816 such that the sealing member 822 stops blocking the shaft port 808 after the end user stops engaging with the engagement surface 816.

The plunger 812 can include a sealing member 822. The sealing member 822 can be attached to a portion of the plunger 812 that is disposed of within the suction chamber 804. The sealing member 822 can be configured to extend into the shaft port 808 to seal against the shaft port 808 and direct the positive pressure pulse 904 (FIG. 9) toward the shaft 112 (FIG. 1).

FIG. 9 illustrates a cross-sectional view of an example of the purging device 800 from FIG. 8 in a working position 902. As discussed herein, moving the purging device 800 from the resting position 820 to the working position 902 can direct the fluid 806 within the suction chamber 804 into the shaft port 808 to transmit the positive pressure pulse 904 toward the distal end of the shaft 112 (FIG. 1).

As discussed with reference to FIG. 8, the plunger 812, and more specifically, the sealing member 822 can be sized such that the sealing member 822 can seal the shaft port 808 to fluidically disconnect the shaft port 808 from the suction chamber 804. The movement of the plunger 812 when moving the purging device 800 from the resting position 820 to the working position 902 can generate the positive pressure pulse 904 and direct it toward the distal end of the shaft 112 (FIG. 1). As such, the positive pressure pulse 904 can dislodge any debris that is clogged, or otherwise blocking, the shaft port 808, the shaft 112 (FIG. 1) or the suction line 122 (FIG. 1) of the endoscopic system 100 (FIG. 1). Moreover, the plunger 812 can be configured such that a portion of the plunger 812 extends within the shaft port 808, which can help break up any clogs, blockages, or the like, that have formed within the shaft port 808.

Any of the purging devices (e.g., the purging device 124, the purging device 300, the purging device 500, or the purging device 800) can be configured to be removably secured to the handle (e.g., the handle 106 (FIG. 1)) and fluidically connected between the suction line (e.g., the suction line 122 (FIG. 1)) and the suction source (e.g., suction source 120 (FIG. 1)). The purging devices can also be configured as an attachment configured to be removably secured to the handle 106 (FIG. 1) and fluidically connected to the suction line (e.g., the suction line 122 (FIG. 1)).

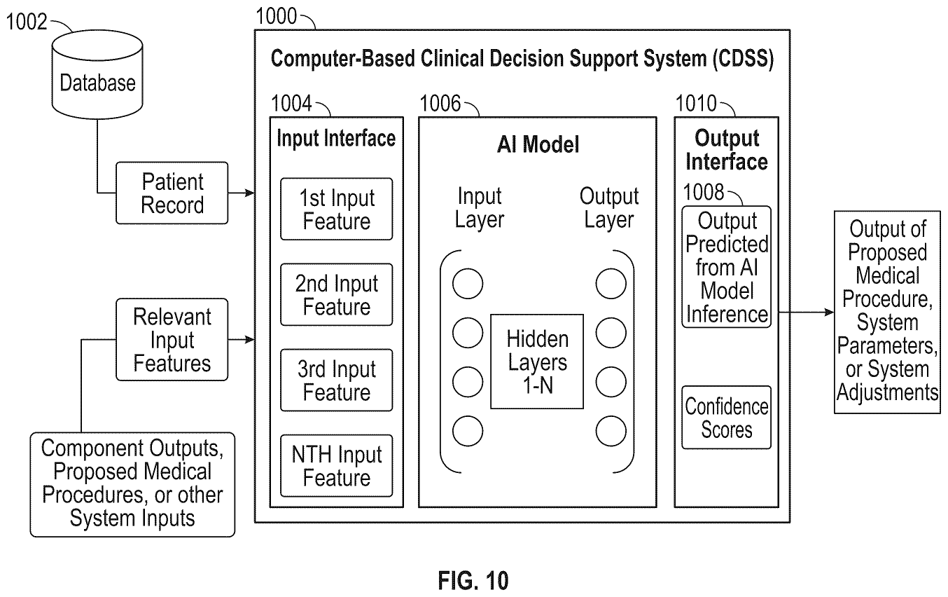

FIG. 10 shows a schematic diagram of an exemplary computer-based clinical decision support system (CDSS) 1000 that is configured to control one or more aspects of the endoscopic system 100, based on input from any one of the components of the endoscopic system (e.g., the inflow pump 118, the suction source 120, the user interface 128, the control module 130, the pressure monitor 132, or the power source 134 (all shown in FIG. 1). The CDSS 1000 can be integral to the control module 130 or can be in communication with the control module 130. In examples, the CDSS 1000 can include an input interface 1004 (e.g., the user interface 128 (FIG. 1) through which medical information, such as, age, weight, sex, which are specific to a patient, or procedure specific information, such as, location of anomaly, planned path for the procedure, planned steps of the procedure, or the like, can be provided as input features to an artificial intelligence (AI) AI model 1006. A processor 1008 (e.g., the control module 130 (FIG. 1)) which performs an inference operation in which the input from any one of the components of the endoscopic system, signals transmitted based on engagement with either of the first engagement member or the second engagement member, medical information, procedure specific information, or the like, are applied to the AI model to generate a suggested medical procedure, and a user interface (UI) through which suggested medical procedure is communicated to a user, e.g., a clinician.

In some embodiments, the input interface 1004 may be a direct data link between the CDSS 1000 and one or more medical devices (e.g., e.g., the endoscopic system 100), that generate at least some of the input features. For example, the input interface 1004 may transmit input from any one of the components of the endoscopic system, medical information, procedure specific information, or the like, directly to the CDSS 1000 during a therapeutic or diagnostic medical procedure. For example, the input interface 1004 may direct data between the CDSS 1000 and a sensor, e.g., the sensor 125, the pressure monitor 132, or the like. Additionally, or alternatively, the input interface 1004 may be a classical user interface that facilitates interaction between a user and the CDSS 1000. For example, the input interface 1004 may facilitate a user interface through which the user may manually enter medical information, procedure specific information, or the like. Additionally, or alternatively, the input interface 1004 may provide the CDSS 1000 with access to an electronic patient record from which one or more input features may be extracted. Such electronic patient records can be stored on a database 1002. In any of these cases, the input interface 1004 can be configured to collect one or more of the following input features in association with a specific patient on or before a time at which the CDSS 1000 is used to assess the safest and most efficient procedure to complete a planned medical procedure.

Based on one or more of the above input features, the processor 1008 performs an inference operation using the AI model 1006 to generate the safest and most efficient medical procedure to perform the medical task. For example, input interface 1004 may deliver any of the medical information, medical procedure information, outputs from any one of the components of the endoscopic system, or signals transmitted based on engagement with either of the first engagement member or the second engagement member into an input layer of the AI model 1006, which propagates these input features through the AI model 1006 to an output layer. The AI model 1006 can provide a computer system the ability to perform tasks, without explicitly being programmed, by making inferences based on patterns found in the analysis of data. The AI model 1006 explores the study and construction of algorithms (e.g., machine-learning algorithms) that may learn from existing data and make predictions about new data. Such algorithms operate by building an AI model from example training data in order to make data-driven predictions or decisions expressed as outputs or assessments.

In examples, the CDSS 1000 can transmit, based on the detected actuation of the purging device 124, an activation summary. The activation summary can be configured to notify the clinician that the purging device 124 is activated and include one or more parameters detected by sensors of the endoscopic system 100, such as the pressure from the pressure monitor 132, a pump rate, e.g., a voltage, from inflow pump 118, a pump rate, e.g., a voltage the suction source 120, or the like. For example, the activation summary can be transmitted to the user interface 128 to provide the clinician with a pressure within the endoscope during the activation of the purging device 124 and a current pressure within the endoscope. The current pressure can be live to show the effectiveness of the activation of the purging device 124.

In examples, the computer-based clinical decision support system (CDSS) 1000 can also be used to control one or more of the inflow pump 118, the suction source 120, the user interface 128, the control module 130, the pressure monitor 132, the power source 134, or the purging device 124) based on any information collected via the components of the endoscopic system 100 or from the patient information provided to the endoscopic system 100 or the computer-based clinical decision support system (CDSS) 1000.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled, and allowing the algorithm to act on that information without guidance. Unsupervised ML is useful in exploratory analysis because it can automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised-ML algorithms are K-means clustering, principal component analysis, and auto-encoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In examples, the AI model may be trained continuously or periodically prior to performance of the inference operation by the processor 1008. Then, during the inference operation, the patient specific input features provided to the AI model may be propagated from an input layer, through one or more hidden layers, and ultimately to an output layer that corresponds to the suggested medical procedure. For example, if the age of the patient, size of the patient, or any other medical information of the patient, and the medical information, such as, the location of the patient indicate the sample may be difficult to obtain, the processor 1008 can suggest a smaller version of the endoscope, suggest a different path that can maximize imaging and sampling efforts, or suggest a maximum energy used for any cutting, ablation, or removal procedures.

During and/or subsequent to the inference operation, the output interface 1010 can transmit any of the safest and most efficient medical procedure may be communicated to the user via the user interface (UI) and/or automatically cause any component of the endoscopic system for performing a desired action. For example, if the imaging quality is poor, the processor 1008 can transmit a signal to the light source control unit to alter the brightness, color, saturation, or any other light parameter, of the light transmitted, send a controlling signal to the fluid source to change a fluid supplied to the pump(s), send a signal to the pump(s) to alter a velocity or volume of fluid supplied to the imaging site, send a signal to the pump(s) to increase or decrease an amount of suction provided to the imaging site. These are exemplary actions that can be taken by the CDSS 1000 to aid in the instruction and procedure of the medical procedure. However, the inventors of the present disclosure have contemplated how the CDSS 1000 can help with any aspect of the medical procedure, such as planning preoperatively, performing intraoperatively, or analyzing the procedure postoperatively, or the like.

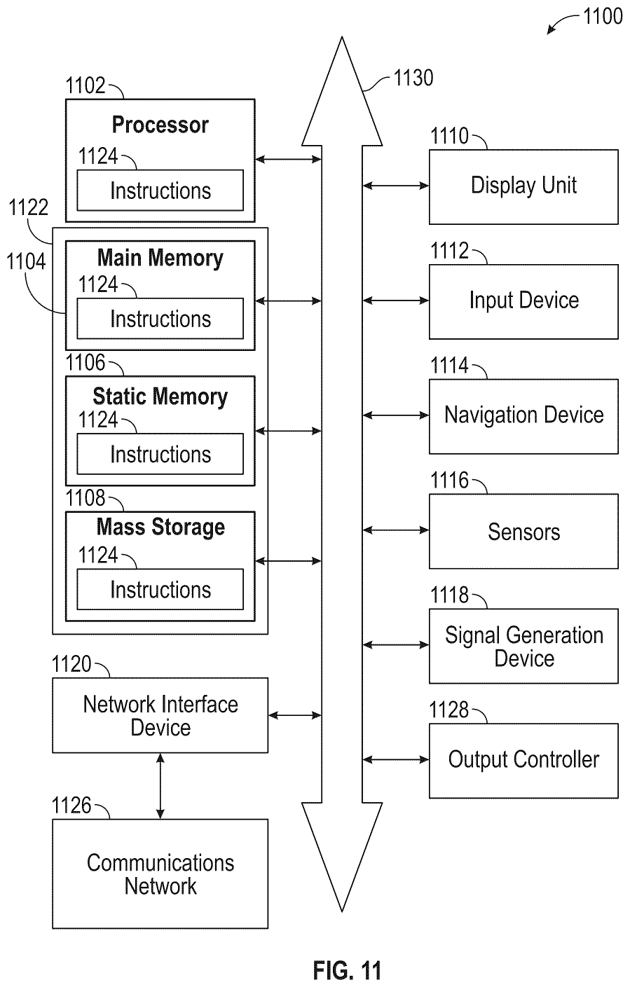

FIG. 11 illustrates a block diagram of an example machine 1100 upon which any one or more of the techniques (e.g., methodologies) discussed herein may perform. Examples, as described herein, may include, or may operate by, logic or a number of components, or mechanisms in the machine 1100. Circuitry (e.g., processing circuitry) is a collection of circuits implemented in tangible entities of the machine 1100 that include hardware (e.g., simple circuits, gates, logic, etc.). Circuitry membership may be flexible over time. Circuitries include members that may, alone or in combination, perform specified operations when operating. In examples, hardware of the circuitry may be immutably designed to carry out a specific operation (e.g., hardwired). In an example, the hardware of the circuitry may include variably connected physical components (e.g., execution units, transistors, simple circuits, etc.), including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In examples, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1100 follow.

In alternative examples, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may act as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein, such as cloud computing, software as a service (Saas), other computer cluster configurations.

The machine 1100 may include a hardware processor 1102 (e.g., a central processing unit (CPU), a graphics processing unit (GPU), a hardware processor core, or any combination thereof), a main memory 1104, a static memory (e.g., memory or storage for firmware, microcode, a basic-input-output (BIOS), and mass storage 1108 (e.g., hard drives, tape drives, flash storage, or other block devices) some or all of which may communicate with each other via an interlink 1130 (e.g., bus). The machine 1100 may further include a display unit 1110, an alphanumeric input device 1112 (e.g., a keyboard), and a user interface (UI) navigation device 1114 (e.g., a mouse). In examples, the display unit 1110, input device 1112 and UI navigation device 1114 may be a touch screen display. The machine 1100 may additionally include a signal generation device 1118 (e.g., a speaker), a network interface device 1120, and one or more sensors 1116, such as a global positioning system (GPS) sensor, compass, accelerometer, or other sensor. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1102, the main memory 1104, the static memory 1106, or the mass storage 1108 may be, or include, a machine-readable medium 1122 on which is stored one or more sets of data structures or instructions 1124 (e.g., software) embodying or utilized by any one or more of the techniques or functions described herein. The instructions 1124 may also reside, completely or at least partially, within any of registers of the processor 1102, the main memory 1104, the static memory 1106, or the mass storage 1108 during execution thereof by the machine 1100. For example, one or any combination of the hardware processor 1102, the main memory 1104, the static memory 1106, or the mass storage 1108 may constitute the machine-readable media 1122. While the machine-readable medium 1122 is illustrated as a single medium, the term “machine-readable medium” may include a single medium or multiple media (e.g., a centralized or distributed database and/or associated caches and servers) configured to store the one or more instructions 1124.

The term “machine-readable medium” may include any medium that is capable of storing, encoding, or carrying instructions for execution by the machine 1100 and that cause the machine 1100 to perform any one or more of the techniques of the present disclosure, or that is capable of storing, encoding or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In examples, information stored or otherwise provided on the machine-readable medium 1122 may be representative of the instructions 1124, such as instructions 1124 themselves or a format from which the instructions 1124 may be derived. This format from which the instructions 1124 may be derived may include source code, encoded instructions (e.g., in compressed or encrypted form), packaged instructions (e.g., split into multiple packages), or the like. The information representative of the instructions 1124 in the machine-readable medium 1122 may be processed by processing circuitry into the instructions to implement any of the operations discussed herein. For example, deriving the instructions 1124 from the information (e.g., processing by the processing circuitry) may include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions 1124.

In examples, the derivation of the instructions 1124 may include assembly, compilation, or interpretation of the information (e.g., by the processing circuitry) to create the instructions 1124 from some intermediate or preprocessed format provided by the machine-readable medium 1122. The information, when provided in multiple parts, may be combined, unpacked, and modified to create the instructions 1124. For example, the information may be in multiple compressed source code packages (or object code, or binary executable code, etc.) on one or several remote servers. The source code packages may be encrypted when in transit over a network and decrypted, uncompressed, assembled (e.g., linked) if necessary, and compiled or interpreted (e.g., into a library, stand-alone executable etc.) at a local machine, and executed by the local machine.

The instructions 1124 may be further transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless data networks (e.g., Institute of Electrical and Electronics Engineers (IEEE) 902.11 family of standards known as Wi-Fi®, IEEE 902.15.4 family of standards, peer-to-peer (P2P) networks, among others. In examples, the network interface device 1120 may include one or more physical jacks (e.g., Ethernet, coaxial, or phone jacks) or one or more antennas to connect to the communications network 1126. In examples, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1100, and includes digital or analog communications signals or other intangible medium to facilitate communication of such software. A transmission medium is a machine-readable medium.

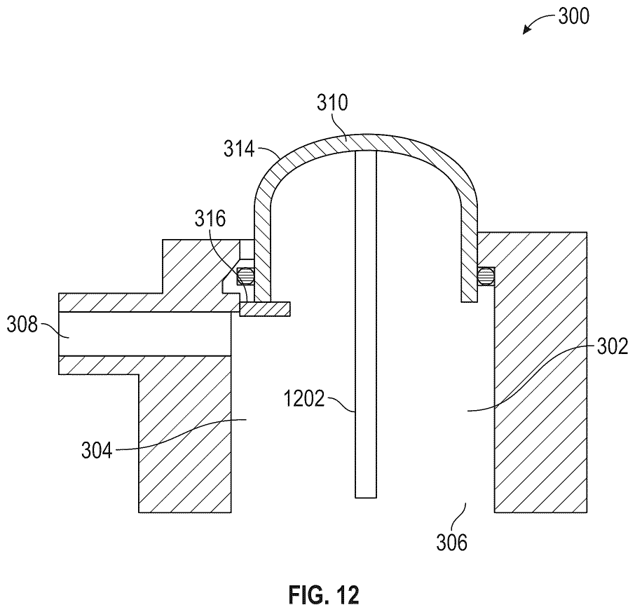

FIG. 12 illustrates a cross-sectional view of an example of a purging device 1200. The purging device 1200 can be implemented as any other purging device disclosed herein, e.g., the purging device 218 (FIG. 2), the purging device 300 (FIG. 3), the purging device 500 (FIG. 5), or the purging device 800 (FIG. 8) and thus can be installed within the handle 106 (FIG. 1). As shown in FIG. 12, the purging device 1200 can include a plumber snake, e.g., a rigid member 1202. The rigid member 1202 can be coupled to an interior surface of the elastic member 310. Thus, as the elastic member 310 is engaged by a clinician the rigid member 1202 can move with the elastic member 310. The movement, e.g., translation, extension, or the like, can cause the rigid member 1202 to engage debris clogged within the inlet 306 to permit fluid flow from the inlet 306 to the outlet 308.

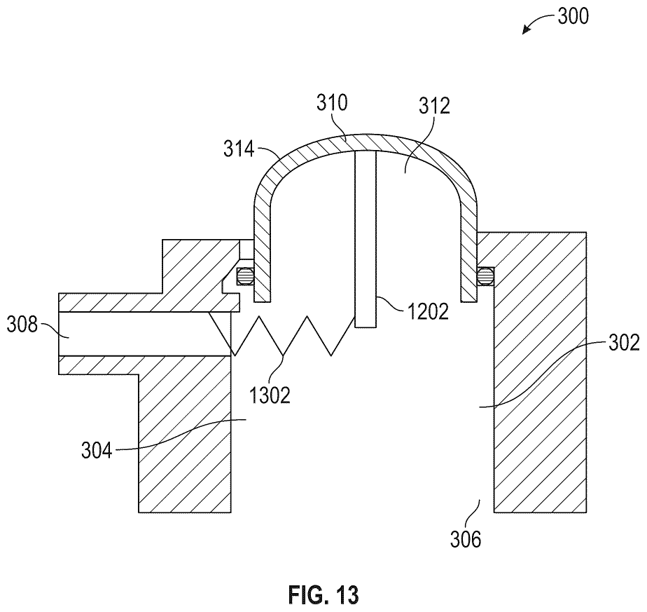

FIG. 13 illustrates a cross-sectional view of an example of a purging device 1300. The purging device 1300 can be implemented as any other purging device disclosed herein, e.g., the purging device 218 (FIG. 2), the purging device 300 (FIG. 3), the purging device 500 (FIG. 5), the purging device 800 (FIG. 8), or the purging device 1200 (FIG. 12), and thus, can be installed within the handle 106 (FIG. 1). As shown in FIG. 13, the purging device 1300 can include a plumber snake, e.g., a rigid member 1202, and a spring element 1302. The rigid member 1202 can be coupled to an interior surface of the elastic member 310 and the spring element 1302 can be coupled to an opposite end of the rigid member 1202. The spring element 1302 can be positioned within the inlet 306 or the outlet 308.

In examples, a first of the spring element 1302 can be positioned within the inlet 306 and a second of the spring element 1302 can be positioned within the outlet 308. As such, the rigid member 1202 can be configured to engage debris within the chamber 302 or to cause disturbances in the fluid within the chamber 302 to dislodge debris buildup in any one or more of the chamber 302, the inlet 306, or the outlet 308. As discussed herein, because the rigid member 1202 can be coupled to an interior surface of the elastic member 310, engagement of the elastic member 310 by the user can cause movement, translation, or the like, of the rigid member 1202 or the spring element 1302.

The following non-limiting examples detail certain aspects of the present subject matter that solve the challenges and provide the benefits discussed herein, among other things.
Example 1 is an endoscope comprising: a handle configured to be held by a clinician to control the endoscope during a medical procedure; a shaft extending from the handle; a suction line routed at least partially within the handle to fluidically connect the shaft to a suction source; and a purging device fluidically connected to the suction line between the suction source and the shaft, the purging device configured to generate a positive pressure pulse to clear debris accumulated within either the shaft or the suction line.
In Example 2, the subject matter of Example 1 optionally includes wherein the purging device comprises: a chamber configured to hold a volume of fluid, the chamber including: an inlet fluidically connected to the shaft; and an outlet fluidically connected to the suction source; a plunger extending into the chamber, the plunger configured to displace the volume of fluid to generate the positive pressure pulse when actuated; and a biasing member configured to return the plunger to a starting position after actuation.
In Example 3, the subject matter of Example 2 optionally includes wherein the plunger comprises: a sealing member attached to a portion of the plunger disposed of within the chamber, the sealing member configured to extend into the outlet to seal against the outlet to direct the positive pressure pulse toward the shaft.
In Example 4, the subject matter of any one or more of Examples 1-3 optionally include wherein the purging device comprises: a fluid chamber configured to hold a fluidic volume, including: a suction inlet fluidically connected to the shaft; and a suction outlet fluidically connected to the suction source; and a flexible diaphragm defining at least a portion of the fluid chamber and configured to displace the fluidic volume when actuated to generate the positive pressure pulse.
In Example 5, the subject matter of Example 4 optionally includes wherein the purging device comprises: a blocking member configured to be engaged by the flexible diaphragm when the flexible diaphragm is actuated to seal the suction outlet and direct the positive pressure pulse through the suction inlet and toward the shaft.
In Example 6, the subject matter of any one or more of Examples 1-5 optionally include wherein the purging device comprises: a hand-actuated pressure bulb fluidically connected to the suction line via a valve, the valve configured to block suction through the suction line when the hand-actuated pressure bulb is actuated such as to direct the positive pressure pulse toward a suction inlet.
In Example 7, the subject matter of any one or more of Examples 1-6 optionally include wherein the purging device is configured as an attachment configured to be removably secured to the handle and fluidically connected to the suction line and the suction source.
In Example 8, the subject matter of any one or more of Examples 1-7 optionally include wherein the purging device is configured as an attachment configured to be removably secured to the handle and fluidically connected to the suction line.
In Example 9, the subject matter of any one or more of Examples 1-8 optionally include wherein the purging device comprises: a suction chamber configured to hold a volume of fluid and fluidically connected to the suction line between the suction source and the shaft, the suction chamber including; a shaft port fluidically connected to the shaft; and a suction port fluidically connected to the suction source; an elastic member engageable to generate the positive pressure pulse within the suction chamber; and a biasing member disposed within the suction chamber and configured to return the elastic member to a starting position after actuation.
In Example 10, the subject matter of Example 9 optionally includes wherein the purging device comprises: an elongated member extending from the elastic member within the suction chamber, the elongated member translates within the suction chamber as the elastic member is engaged such that the elongated member blocks the suction port to direct the positive pressure pulse through the shaft port and toward the shaft.
In Example 11, the subject matter of any one or more of Examples 1-10 optionally include wherein the endoscope is a lithotripter configured to remove debris from within a kidney of a patient.
In Example 12, the subject matter of any one or more of Examples 1-11 optionally include wherein the endoscope comprises: a console including control circuitry for controlling operation of the endoscope during a medical procedure; a sensor configured to detect an actuation of the purging device.
In Example 13, the subject matter of Example 12 optionally includes wherein the controlling circuitry is configured to: transmit, based on the detected actuation of the purging device, an activation summary to notify the clinician that the purging device was activated.
In Example 14, the subject matter of Example 13 optionally includes wherein the activation summary includes a first pressure relative to a pressure within the endoscope during the activation of the purging device and a second pressure relative to a current pressure within the endoscope.
Example 15 is a purging device for a medical instrument, the medical instrument including a handle, a shaft extending from the handle, and suction tubing extending between the handle and a suction source, the purging device comprising: a housing including: a chamber defining a volume of fluid; and an attachment interface configured to attach the purging device to the handle of the medical instrument such that the chamber fluidically connects the suction source and the shaft; and an engagement member engageable from an exterior of the housing, the engagement member configured to generate a positive pressure pulse within the chamber to energize the volume of fluid from the chamber and unclog debris from either one of a suction tubing or the shaft of the medical instrument.
In Example 16, the subject matter of Example 15 optionally includes wherein the housing comprises: a suction source port configured to connect to the suction source; and a suction tubing port configured to connect to the suction tubing to fluidically connect the purging device and the shaft of the medical instrument.
In Example 17, the subject matter of Example 16 optionally includes a plunger extending into the chamber, the plunger configured to displace the volume of fluid to generate the positive pressure pulse when actuated; and a biasing member configured to return the plunger to a starting position after actuation.
In Example 18, the subject matter of Example 17 optionally includes wherein the plunger comprises: a sealing member attached to a portion of the plunger disposed of within the chamber, the sealing member configured to extend into the suction tubing port to direct the positive pressure pulse through the suction tubing port and toward the shaft.
In Example 19, the subject matter of any one or more of Examples 16-18 optionally include wherein the engagement member includes a flexible diaphragm defining at least a of the chamber and configured to displace the volume of fluid when actuated to generate the positive pressure pulse.
In Example 20, the subject matter of Example 19 optionally includes a blocking member configured to be engaged by the flexible diaphragm when the flexible diaphragm is actuated to seal the suction source port and direct the positive pressure pulse through the suction tubing port and toward the shaft.
Example 21 is a method, system, or apparatus including any element of any of Examples 1-20.

The above-detailed description includes references to the accompanying drawings, which form a part of the detailed description. The drawings show, by way of illustration, specific examples that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other examples may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The device may also be sterilized using any other technique known in the art, including but limited to beta or gamma radiation, ethylene oxide, or steam.

What is claimed is:

1. An endoscope comprising:
a handle configured to be held by a clinician to control the endoscope during a medical procedure;
a shaft extending from the handle;
a suction line routed at least partially within the handle to fluidically connect the shaft to a suction source; and
a purging device fluidically connected to the suction line between the suction source and the shaft, the purging device configured to generate a positive pressure pulse to clear debris accumulated within either the shaft or the suction line.

2. The endoscope of claim 1, wherein the purging device comprises:
a chamber configured to hold a volume of fluid, the chamber including:
an inlet fluidically connected to the shaft; and
an outlet fluidically connected to the suction source;
a plunger extending into the chamber, the plunger configured to displace the volume of fluid to generate the positive pressure pulse when actuated; and
a biasing member configured to return the plunger to a starting position after actuation.

3. The endoscope of claim 2, wherein the plunger comprises:
a sealing member attached to a portion of the plunger disposed of within the chamber, the sealing member configured to extend into the outlet to seal against the outlet to direct the positive pressure pulse toward the shaft.

4. The endoscope of claim 1, wherein the purging device comprises:
a fluid chamber configured to hold a fluidic volume, including:
a suction inlet fluidically connected to the shaft; and
a suction outlet fluidically connected to the suction source; and
a flexible diaphragm defining at least a portion of the fluid chamber and configured to displace the fluidic volume when actuated to generate the positive pressure pulse.

5. The endoscope of claim 4, wherein the purging device comprises:
a blocking member configured to be engaged by the flexible diaphragm when the flexible diaphragm is actuated to seal the suction outlet and direct the positive pressure pulse through the suction inlet and toward the shaft.

6. The endoscope of claim 1, wherein the purging device comprises:
a hand-actuated pressure bulb fluidically connected to the suction line via a valve, the valve configured to block suction through the suction line when the hand-actuated pressure bulb is actuated such as to direct the positive pressure pulse toward a suction inlet.

7. The endoscope of claim 1, wherein the purging device is configured as an attachment configured to be removably secured to the handle and fluidically connected to the suction line and the suction source.

8. The endoscope of claim 1, wherein the purging device is configured as an attachment configured to be removably secured to the handle and fluidically connected to the suction line.

9. The endoscope of claim 1, wherein the purging device comprises:
a suction chamber configured to hold a volume of fluid and fluidically connected to the suction line between the suction source and the shaft, the suction chamber including;
a shaft port fluidically connected to the shaft; and
a suction port fluidically connected to the suction source;
an elastic member engageable to generate the positive pressure pulse within the suction chamber; and
a biasing member disposed within the suction chamber and configured to return the elastic member to a starting position after actuation.

10. The endoscope of claim 9, wherein the purging device comprises:
an elongated member extending from the elastic member within the suction chamber, the elongated member translates within the suction chamber as the elastic member is engaged such that the elongated member blocks the suction port to direct the positive pressure pulse through the shaft port and toward the shaft.

11. The endoscope of claim 1, wherein the endoscope is a lithotripter configured to remove debris from within a kidney of a patient.

12. The endoscope of claim 1, wherein the endoscope comprises:
a console including control circuitry for controlling operation of the endoscope during a medical procedure;
a sensor configured to detect an actuation of the purging device.

13. The endoscope of claim 12, wherein the controlling circuitry is configured to:
transmit, based on the detected actuation of the purging device, an activation summary to notify the clinician that the purging device was activated.

14. The endoscope of claim 13, wherein the activation summary includes a first pressure relative to a pressure within the endoscope during the activation of the purging device and a second pressure relative to a current pressure within the endoscope.

15. A purging device for a medical instrument, the medical instrument including a handle, a shaft extending from the handle, and suction tubing extending between the handle and a suction source, the purging device comprising:
a housing including:
a chamber defining a volume of fluid; and
an attachment interface configured to attach the purging device to the handle of the medical instrument such that the chamber fluidically connects the suction source and the shaft; and
an engagement member engageable from an exterior of the housing, the engagement member configured to generate a positive pressure pulse within the chamber to energize the volume of fluid from the chamber and unclog debris from either one of a suction tubing or the shaft of the medical instrument.

16. The purging device of claim 15, wherein the housing comprises:
a suction source port configured to connect to the suction source; and
a suction tubing port configured to connect to the suction tubing to fluidically connect the purging device and the shaft of the medical instrument.

17. The purging device of claim 16, comprising:
a plunger extending into the chamber, the plunger configured to displace the volume of fluid to generate the positive pressure pulse when actuated; and
a biasing member configured to return the plunger to a starting position after actuation.

18. The purging device of claim 17, wherein the plunger comprises:
a sealing member attached to a portion of the plunger disposed of within the chamber, the sealing member configured to extend into the suction tubing port to direct the positive pressure pulse through the suction tubing port and toward the shaft.

19. The purging device of claim 16, wherein the engagement member includes a flexible diaphragm defining at least a of the chamber and configured to displace the volume of fluid when actuated to generate the positive pressure pulse.

20. The purging device of claim 19, comprising:
a blocking member configured to be engaged by the flexible diaphragm when the flexible diaphragm is actuated to seal the suction source port and direct the positive pressure pulse through the suction tubing port and toward the shaft.
